(19) United States
(12) Patent Application Publication
(10) Pub. No.: US 2019/0365243 A1
(43) Pub. Date: Dec. 5, 2019

(54) BLOOD-PRESSURE-RELATED INFORMATION DISPLAY APPARATUS AND METHOD

(71) Applicants: OMRON CORPORATION, Kyoto-shi (JP); OMRON HEALTHCARE CO., LTD., Muko-shi (JP)

(72) Inventors: Kosuke INOUE, Kyoto (JP); Shusuke ESHITA, Kyoto (JP)

(73) Assignees: OMRON CORPORATION, Kyoto-shi (JP); OMRON HEALTHCARE CO., LTD., Muko-shi (JP)

(21) Appl. No.: 16/542,519

(22) Filed: Aug. 16, 2019

Related U.S. Application Data
(63) Continuation of application No. PCT/JP2018/009576, filed on Mar. 12, 2018.

(30) Foreign Application Priority Data
Mar. 14, 2017 (JP) 2017-048759

(51) Int. Cl. A61B 5/022; A61B 5/00

(52) U.S. Cl. CPC A61B 5/022; A61B 5/02108; A61B 5/742

(57) ABSTRACT

An apparatus of an embodiment is an apparatus that displays information regarding blood pressure of a measurement subject on a display screen and includes an information acquisition unit that acquires surge blood pressure information of the measurement subject; and a display processing unit that displays the surge blood pressure information as a circle graph on the display screen, the circle graph having a circular time axis corresponding to twenty-four hours, having a time scale indicating elapse of a unit time in the twenty-four hours clockwise along the time axis, and including an aggregation of bars of a bar graph that extends outward in a radial direction of an arc of the time axis and has height corresponding to a value based on the surge blood pressure information per the unit time.

### CROSS REFERENCE TO RELATED APPLICATIONS

This application is a Continuation Application of PCT Application No. PCT/JP2018/009576, filed Mar. 12, 2018 and based upon and claiming the benefit of priority from Japanese Patent Application No. 2017-048759, filed Mar. 14, 2017, the entire contents of all of which are incorporated herein by reference.

### FIELD

The present invention relates to a blood-pressure-related information display apparatus and a blood-pressure-related information display method and more specifically relates to a blood-pressure-related information display apparatus and a blood-pressure-related information display method for making a graph of information regarding blood pressure of a measurement subject and displaying the graph on a display screen.

### BACKGROUND

Conventionally, as disclosed in, for example, Jpn. Pat. Appln. KOKAI Publication No. 2004-261452, there has been proposed, as an apparatus that displays information related to blood pressure of a measurement subject, a sphygmomanometer that stores a measured blood pressure value on a memory in association with measurement time information and a measurement condition, calculates an average value obtained by averaging blood pressure values measured a plurality of times in a specified period of time, calculates a risk value based on the calculation result, and displays the risk value.

Further, in recent years, it has been proposed that blood pressure of a measurement subject be measured by a portable sphygmomanometer at predetermined time intervals for a long period of time (e.g., twenty-four hours) and health of the measurement subject be managed based on the measurement result.

### SUMMARY

For example, in a case where blood pressure values measured at predetermined time intervals for a long period of time are presented in time series in the form of numerical values to a user (a measurement subject or a third party such as a family member of the measurement subject or a doctor in charge of the measurement subject), the user needs to analyze a tendency for blood pressure of the measurement subject based on an enormous number of numerical values. This may put a heavy burden on the user. Further, in a case where the user wrongly determines the tendency for the blood pressure of the measurement subject, it is impossible to accurately manage health of the measurement subject. This may deteriorate his/her health status.

It is expected that, in a case where a result of 24-hour measurement of the blood pressure is presented to the user, the user can accurately and easily grasp the health status of the measurement subject based on, for example, a period of time in which the blood pressure of the measurement subject becomes high, presence/absence of surge blood pressure that is sharply rising blood pressure of the measurement subject, and a relation between an occurrence timing of atrial fibrillation (AF) and a blood pressure value and can assist in health management of the measurement subject.

The present invention has been made in view of the above-mentioned circumstances, and an object thereof is to provide a blood-pressure-related information display apparatus and a blood-pressure-related information display method that facilitate a grasp of a health status of a measurement subject based on blood-pressure-related information that is continuously measured for a long period of time.

To overcome above issue, the first aspect of the present invention is a blood-pressure-related information display that displays information regarding blood pressure of a measurement subject on a display screen. The blood-pressure-related information display apparatus comprising: an information acquisition unit that acquires surge blood pressure information of the measurement subject; and a display processing unit that displays the surge blood pressure information as a circle graph on the display screen, the circle graph having a circular time axis corresponding to twenty-four hours, having a time scale indicating elapse of a unit time in the twenty-four hours clockwise along the time axis, and including an aggregation of bars of a bar graph that extends outward in a radial direction of an arc of the time axis and has height corresponding to a value based on the surge blood pressure information per the unit time.

The second aspect of the present invention according to the first aspect of the present invention, wherein the display processing unit divides the bar graph into a plurality of regions in the radial direction and shows the plurality of regions with different display colors.

The third aspect of the present invention according to the first or second aspect of the present invention, wherein the information acquisition unit further acquires atrial fibrillation occurrence information of the measurement subject, and the display processing unit displays a plot on the time scale corresponding to a time at which atrial fibrillation of the measurement subject has occurred.

The fourth aspect of the present invention according to any one of the first to third aspect of the present invention, wherein the surge blood pressure information includes a systolic surge blood pressure value of the measurement subject in the twenty-four hours, and the display processing unit displays a bar of the bar graph corresponding to the systolic surge blood pressure value with color different from color of another bar of the bar graph.

The fifth aspect of the present invention according to any one of the first to fourth aspect of the present invention, further comprising: a strip that is to be worn on a measurement part of the measurement subject; a sensor unit that is provided so that at least a part of the sensor unit is brought into contact with the measurement part when the strip is worn on the measurement part; a blood-pressure-information measurement unit that adjusts pressure to be applied from the sensor unit to the measurement part and measures systolic blood pressure and diastolic blood pressure of the measurement subject based on the pressure; and a communication unit that transmits values measured by the blood-pressure-information measurement unit to outside.

According to the first aspect of the present invention, blood-pressure-related information that is continuously measured for a long period of time is displayed as the circle graph. This makes it possible to easily grasp a health status of the measurement subject.

According to the second aspect of the present invention, the display color of the bar graph is gradually changed. This allows a user to distinguish a rise and fall in surge blood pressure based on color and therefore to easily recognize the health status of the measurement subject.

According to the third aspect of the present invention, the time at which (or a period of time in which) atrial fibrillation has occurred is displayed. This makes it possible to recognize, for example, a period of time in which a surge blood pressure value and atrial fibrillation of the measurement subject occur and therefore to reduce a burden of health management of the measurement subject and assist in improving the health status of the measurement subject.

According to the fourth aspect of the present invention, the surge blood pressure value of the measurement subject and the time at which (or a period of time in which) atrial fibrillation has occurred are displayed in association with each other. This allows the user to easily recognize an interrelationship between surge blood pressure and atrial fibrillation and therefore to reduce a burden of the health management of the measurement subject and assist in improving the health status of the measurement subject.

According to the fifth aspect of the present invention, the measurement subject can check his/her health status by using an instrument that is always worn on a measurement part in order to measure blood pressure. This makes it possible to further improve convenience for the measurement subject.

### BRIEF DESCRIPTION OF THE DRAWING(S)

FIG. 1 schematically illustrates the whole configuration of a blood-pressure-related information management system including a blood-pressure-related information display apparatus according to an embodiment of the present invention.

FIG. 2 is a block diagram illustrating an example of a configuration of the blood pressure measuring instrument illustrated in FIG. 1.

FIG. 3 is a block diagram illustrating an example of a configuration of the user terminal illustrated in FIG. 1.

FIG. 4 is a block diagram illustrating an example of a configuration of the server illustrated in FIG. 1.

FIG. 5 is a flowchart showing a blood-pressure-related information display method according to an embodiment of the present invention.

FIG. 6 is a plan view illustrating an example where blood-pressure-related information is displayed on the display unit of the user terminal.

FIG. 7 is an explanatory diagram illustrating a part of a circle graph included in a display of blood-pressure-related information.

FIG. 8 is a flow-chart illustrating another example of the blood-pressure-related information display method according to an embodiment of the present invention.

FIG. 9 is a plan view illustrating an example of a display of the blood-pressure-related information in the blood pressure measuring instrument.

### DETAILED DESCRIPTION

Hereinafter, embodiments according to the present invention will be described with reference to the drawings.

### Embodiment

(Configuration)

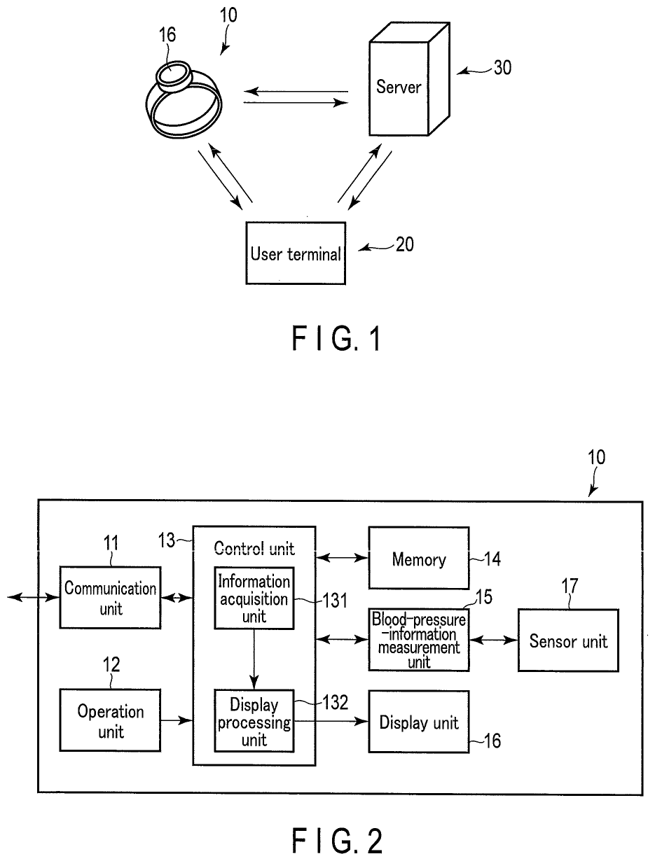

FIG. 1 schematically illustrates the whole configuration of a blood-pressure-related information management system including a blood-pressure-related information display apparatus according to an embodiment of the present invention.

The blood-pressure-related information management system includes a blood pressure measuring instrument 10, a user terminal 20, and a server 30. The blood pressure measuring instrument 10, the user terminal 20, and the server 30 are configured to communicate with each other over a network via wireless communication or wired communication. Note that the blood pressure measuring instrument 10 may be configured to communicate with the server 30 via the user terminal 20.

The blood pressure measuring instrument 10 includes a strip and a measuring instrument main body provided in a casing. A measurement subject wears the blood pressure measuring instrument 10 by winding the strip around a measurement part so that at least a part of the measuring instrument main body is in contact with a position (measurement part) inside which an artery (e.g., radial artery) serving as a target whose blood pressure is to be measured exists and operates the measuring instrument main body to thereby measure and display blood-pressure-related information as described below.

For example, the blood pressure measuring instrument 10 can measure the blood-pressure-related information of the measurement subject by employing a continuous measuring blood pressure detection method using a PTT method, a tonometry method, an optical method, a radio wave method, an ultrasonic method, or the like. The PTT method is a method in which pulse transmit time (PTT) is measured and a blood pressure value is estimated based on the measured pulse transmit time. The tonometry method is a method in which a pressure sensor is directly brought into contact with a part (measurement part) of a living body through which an artery such as a radial artery of a wrist passes and a blood pressure value is measured by using information detected by the pressure sensor. The optical method, the radio wave method, and the ultrasonic method are methods in which a blood vessel is exposed to light, a radio wave, or an ultrasonic wave and a blood pressure value is measured based on a reflected wave thereof.

The user terminal 20 can be, for example, a mobile communication terminal such as a smartphone, a tablet, or a mobile phone or a communication terminal such as a personal computer.

Note that, although FIG. 1 illustrates only a single blood pressure measuring instrument 10 and a single user terminal 20, the server 30 can communicate with a plurality of blood pressure measuring instruments 10 and a plurality of user terminals 20 and can manage pieces of blood-pressure-related information measured by the plurality of blood pressure measuring instruments 10.

FIG. 2 is a block diagram illustrating an example of a configuration of the blood pressure measuring instrument illustrated in FIG. 1.

The blood pressure measuring instrument 10 includes a communication unit 11, an operation unit 12, a control unit 13, a memory 14, a blood-pressure-information measurement unit 15, a display unit 16, and a sensor unit 17.

The communication unit 11 is controlled by the control unit 13 and can transmit predetermined information to the outside over a network and can receive information from the outside and transmit the information to the control unit 13. Communication performed by the communication unit 11 over the network may be wireless communication or wired communication. In the present embodiment, the network is, for example, the Internet but is not limited to thereto. The network may be another type of network such as a local area network (LAN) or may be one-to-one communication using a communication cable such as an USB cable.

In response to operation performed by a user (measurement subject) on an operation device (not illustrated) such as a touchscreen, an operation button, or an operation key, the operation unit 12 outputs an operation signal based on the operated content to the control unit 13. Further, the operation unit 12 is not limited to the touchscreen, the operation button, or the operation key and may include, for example, a speech recognition unit that recognizes an operation instruction spoken by the user, a biometric authentication unit that authenticates a part of a living body of the user, and an image recognition unit that recognizes expression and gesture of the user based on an image obtained by imaging means.

The memory 14 stores data of a program for controlling the blood pressure measuring instrument 10, setting data for setting various functions of the blood pressure measuring instrument 10, data of a measurement result, and the like. Further, the memory 14 is used as a work memory or the like when a program is executed.

The sensor unit 17 is provided at a position at which at least a part thereof is brought into contact with a measurement part of the measurement subject when the strip of the blood pressure measuring instrument 10 is worn on the measurement part and detects a pressure pulse wave of the measurement subject. The sensor unit 17 includes, for example, an air bag, a pressing portion, a pump, and at least one pressure sensor and can adjust a pressure of the pressing portion for pressing the measurement part of the measurement subject by adjusting an air volume inside the air bag by using the pump. At this time, for example, pressing force applied by the sensor unit 17 to a radial artery of the measurement subject is equal to an internal pressure of the air bag. Note that the sensor unit 17 only needs to be configured to press the measurement part of the measurement subject and is not limited to a configuration including an air bag.

The at least one pressure sensor is arranged at a position at which the pressing portion and the measurement part are brought into contact with each other. The pressure sensor can include, for example, a pressure-sensitive diode formed on a surface of the pressing portion that is to be brought into contact with the measurement part.

The pressure sensor is placed at, for example, a position at which the radial artery of the measurement subject is pressed so as to extend across (substantially orthogonal to) the radial artery and can detect a pressure oscillation wave, i.e., a pressure pulse wave generated from the radial artery of the measurement subject to be transmitted to skin.

The blood-pressure-information measurement unit 15 adjusts pressure of the air bag of the sensor unit 17 by controlling the pump based on a control signal from the control unit 13 and receives a pressure pulse wave detected by the sensor unit 17, thereby measuring systolic blood pressure (SBP) and diastolic blood pressure (DBP) of the measurement subject. Further, the blood-pressure-information measurement unit 15 can measure a pulse rate of the measurement subject based on the pressure pulse wave obtained from the sensor unit 17. The blood-pressure-information measurement unit 15 outputs the pressure pulse wave, measurement values of the measured blood pressure, and the measured pulse rate to the control unit 13.

Note that the sensor unit 17 is not limited to the above-mentioned configuration and may be an optical sensor or an ultrasonic sensor. Further, the sensor unit 17 may measure, for example, pulse transmit time (PTT) between two electrodes pressed against the radial artery. In this case, the sensor unit 17 transmits the PTT to the blood-pressure-information measurement unit 15, and the blood-pressure-information measurement unit 15 can measure, for example, systolic blood pressure (SBP) and diastolic blood pressure (DBP) of the measurement subject based on the PTT measured by the sensor unit 17, a distance between the two electrodes, and pressing force to the measurement part.

The display unit 16 includes, for example, a display screen (e.g., liquid crystal display (LCD), electroluminescence (EL) display, or the like), an indicator, and the like and displays predetermined information in accordance with a control signal from the control unit 13. The control unit 13 includes at least one processor such as a central processing unit (CPU) or a micro processing unit (MPU) and can control the memory 14, the display unit 16, and the communication unit 11 in accordance with a program stored on the memory 14 based on an operation signal from the operation unit 12 and a measurement result from the blood-pressure-information measurement unit 15. For example, in response to an instruction from the operation unit 12, the control unit 13 can execute a mode in which current blood pressure of the measurement subject is measured. Further, in response to an instruction from the operation unit 12, the control unit 13 can execute a mode in which blood pressure and the like (systolic blood pressure, diastolic blood pressure, and the like) of the measurement subject are automatically and continuously measured for a long period of time (e.g., twenty-four hours) at predetermined time intervals (e.g., for each beat of a pulse).

The control unit 13 may include an information acquisition unit 131 and a display processing unit 132.

The information acquisition unit 131 can acquire, via the communication unit 11, surge blood pressure information and atrial fibrillation occurrence information based on blood-pressure-related information of the measurement subject.

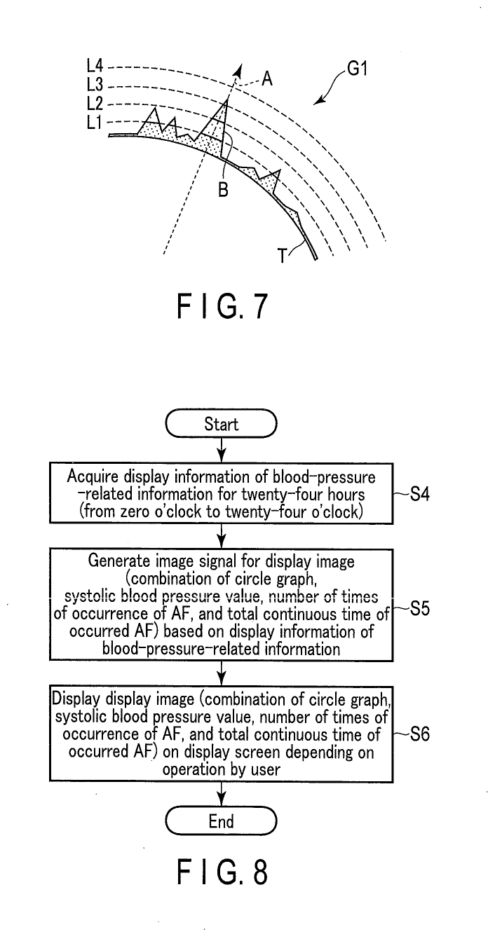

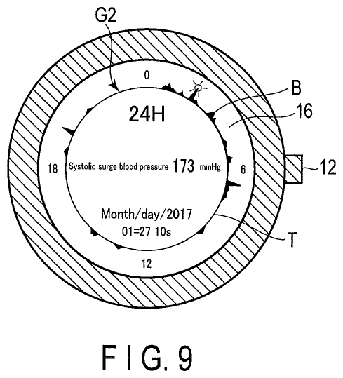

The display processing unit 132 receives the surge blood pressure information from the information acquisition unit 131 and, as illustrated in, for example, FIG. 9, can display the surge blood pressure information as a circle graph G2 on the display screen of the display unit 16, the circle graph G2 having a circular time axis T corresponding to twenty-four hours, having a time scale indicating elapse of a unit time in twenty-four hours clockwise along the time axis T, and including an aggregation of bars of a bar graph B that extends outward in a radial direction A (illustrated in FIG. 7) of an arc of the time axis T and has height based on a surge blood pressure value per unit time.

Further, the display processing unit 132 can receive the atrial fibrillation occurrence information from the information acquisition unit 131 and display a plot on the time scale of the time axis T corresponding to a time at which atrial fibrillation of the measurement subject has occurred or display a bar of the bar graph B at the time at which the atrial fibrillation has occurred with color different from that of another bar of the bar graph B.

Note that the display processing unit 132 can generate an image signal that causes various images (including a still image and a moving image) in addition to the above-mentioned circle graph G2 to be displayed on the display screen of the display unit 16 based on information and an instruction obtained from the communication unit 11, the operation unit 12, and the memory 14. The display processing unit 132 outputs the generated image signal to the display unit 16.

Note that the blood pressure measuring instrument 10 may have at least one of an activity measuring function, a step counting function, a sleep state measuring function, and an environment (temperature, humidity) measuring function. That is, the blood pressure measuring instrument 10 may further include at least one of a triaxial sensor, an environment sensor, and a barometer and may be configured to detect acceleration by using the triaxial sensor so as to calculate activity of the measurement subject, may be configured to detect acceleration by using the triaxial sensor and also detect atmospheric pressure by using the barometer so as to calculate the number of steps of the measurement subject, the number of steps obtained when the measurement subject walks fast, the number of steps obtained when the measurement subject goes up stairs, and the like, or may be configured to detect acceleration by using the triaxial sensor and detect a state in which the measurement subject turns over in his/her sleep so as to detect his/her sleep state.

Further, the environment sensor includes a sensor that measures information regarding an environment around the user and acquires measured environment data. The environment sensor may include sensors that measure an air temperature, a temperature, humidity, sound, light, and the like. The environment sensor may include a sensor that measures information regarding an environment (environment data) that is expected to directly or indirectly relate to a change in blood pressure value. Further, the control unit 13 can associate measurement data measured by the environment sensor with a measurement time set based on time information and acquire the measurement data (environment data).

For example, an air temperature (change in air temperature) is considered to be one of elements that may cause a change in blood pressure of a human being. Therefore, air temperature data measured by the environment sensor is information that may be a factor in a change in blood pressure of the user and can be output as measurement data of a single element other than blood pressure. Further, in a case where not only an air temperature but also humidity, sound, light, and the like are considered to be one of elements that may cause a change in blood pressure, the measurement data thereof may be output as measurement data of elements other than blood pressure.

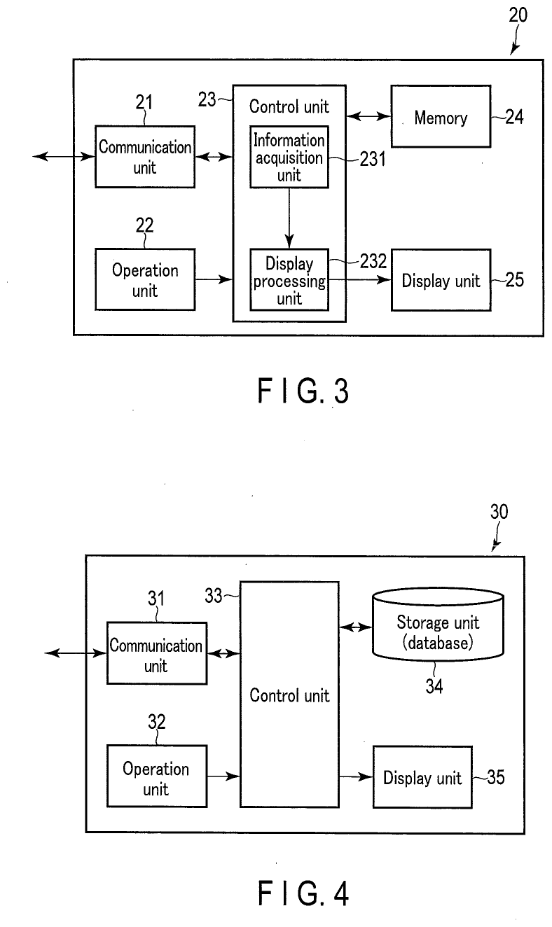

FIG. 3 is a block diagram illustrating an example of a configuration of the user terminal illustrated in FIG. 1. The user terminal 20 includes a communication unit 21, an operation unit 22, a control unit 23, a memory 24, and a display unit 25.

In the present embodiment, the user terminal 20 is, for example, a mobile communication terminal such as a smartphone or a tablet and is a terminal in which application software (program) is installed so that processing described below is executable.

The communication unit 21 is controlled by the control unit 23 and can transmit predetermined information to the outside over a network and can receive information from the outside and transmit the information to the control unit 23. Communication performed by the communication unit 21 over the network may be wireless communication or wired communication. In the present embodiment, the network is, for example, the Internet but is not limited to thereto. The network may be another type of network such as a local area network (LAN) or may be one-to-one communication using a communication cable such as an USB cable.

The operation unit 22 includes, for example, a touchscreen and an operation button and transmits an operation signal corresponding to operation performed by the user (e.g., measurement subject) to the control unit 23. Further, the operation unit 22 is not limited to the touchscreen or the operation button and may include, for example, operation means such as a keyboard and a mouse, a speech recognition unit that recognizes an operation instruction spoken by the user, a biometric authentication unit that authenticates a part of the living body of the user, and an image recognition unit that recognizes expression and gesture of the user based on an image obtained by imaging means.

The memory 24 includes, for example, a random access memory (RAM) that is used as a work area necessary to execute a program in the control unit 23 and a read only memory (ROM) for storing a basic program to be executed in the control unit 23. Further, a semiconductor memory (memory card, solid state drive (SSD)) or the like may be provided as a storage medium of an auxiliary storage apparatus for reinforcing a storage area of the memory 24.

The display unit 25 includes a display screen (e.g., LCD or EL display). The display unit 25 is controlled by the control unit 23 and can display a predetermined image on the display screen.

The control unit 23 includes, for example, at least one processor such as a CPU or an MPU and an auxiliary circuit thereof and can control each configuration of the user terminal 20. The control unit 23 executes processing described below in accordance with a program and data stored on the memory 24. That is, the control unit 23 can process data input from the operation unit 22 and the communication unit 21 and cause the processed data to be stored on the memory 24, to be displayed on the display unit 25, or to be output from the communication unit 21.

The control unit 23 may include an information acquisition unit 231 and a display processing unit 232.

The information acquisition unit 231 can acquire, via the communication unit 21, surge blood pressure information and atrial fibrillation occurrence information based on blood-pressure-related information of the measurement subject.

The display processing unit 232 receives the surge blood pressure information from the information acquisition unit 231 and, as illustrated in, for example, FIGS. 6 and 7, can display the surge blood pressure information as a circle graph G1 on the display screen of the display unit 25, the circle graph G1 having a circular time axis T corresponding to twenty-four hours, having a time scale indicating elapse of a unit time in twenty-four hours clockwise along the time axis T, and including an aggregation of bars of a bar graph B that extends outward in a radial direction A of an arc of the time axis T and has height based on a surge blood pressure value per unit time.

Further, the display processing unit 232 can receive the atrial fibrillation occurrence information from the information acquisition unit 231 and display a plot on the time scale of the time axis T corresponding to a time at which atrial fibrillation of the measurement subject has occurred or display a bar of the bar graph B at the time at which the atrial fibrillation has occurred with color different from that of another bar of the bar graph B.

Note that the display processing unit 232 can generate an image signal that causes various images (including a still image and a moving image) in addition to the above-mentioned circle graph G1 to be displayed on the display screen of the display unit 25 based on information and an instruction obtained from the communication unit 21, the operation unit 22, and the memory 24. The display processing unit 232 outputs the generated image signal to the display unit 25.

FIG. 4 is a block diagram illustrating an example of a configuration of the server illustrated in FIG. 1.

The server 30 includes a communication unit 31, an operation unit 32, a control unit 33, a storage unit 34, and a display unit 35. The server 30 is, for example, a general computer in which a program (software) is installed so as to perform processing described below.

The operation unit 32 includes, for example, a keyboard and a mouse and transmits an operation signal showing operation performed by the user to the control unit 33. Further, the operation unit 32 may include another operation device such as a touchscreen, instead of or in addition to the keyboard and the mouse.

The communication unit 31 transmits information from the control unit 33 to the outside (in this example, the blood pressure measuring instrument 10 or the user terminal 20) over the network and receives information transmitted from the outside and transmits the information to the control unit 33 over the network.

The storage unit 34 includes a RAM that is used as a work area necessary to execute a program in the control unit 33 and a ROM for storing a basic program to be executed in the control unit 33. The storage unit 34 includes a database including data of a large number of pieces of blood-pressure-related information transmitted from the blood pressure measuring instrument 10 or the user terminal 20. Further, a magnetic disk (hard disk (HD), flexible disk (FD)), an optical disc (compact disc (CD), digital versatile disk (DVD), a Blu-ray (registered trademark) Disc (BD)), a magneto-optical disk (MO), a semiconductor memory (memory card, SSD), or the like may be used as a storage medium of an auxiliary storage apparatus for reinforcing a storage area of the storage unit 34.

The display unit 35 includes a display screen (e.g., LCD or EL display). The display unit 35 is controlled by the control unit 33 and displays a predetermined image (including a still image and a moving image) on the display screen.

The control unit 33 includes, for example, at least one processor such as a CPU or an MPU and an auxiliary circuit thereof. The control unit 33 can control each unit of the server 30, execute predetermined processing in accordance with a program and data stored on the storage unit 34, process data input from the operation unit 32 and the communication unit 31, and cause the processed data to be stored on the storage unit 34, to be displayed on the display unit 35, or to be output from the communication unit 31.

(Operation)

Next, an example of operation of the blood-pressure-related information management system including the blood-pressure-related information display apparatus configured as described above will be described.

First, an example of operation in which blood-pressure-related information measured by the blood pressure measuring instrument 10 is accumulated in the storage unit 34 of the server 30 will be described.

When the user operates the operation unit 12 of the blood pressure measuring instrument 10 so as to execute a mode in which blood-pressure-related information is automatically and continuously measured for a long period of time, the control unit 13 of the blood pressure measuring instrument 10 controls the blood-pressure-information measurement unit 15 and the sensor unit 17 so as to measure, for example, blood-pressure-related information including systolic blood pressure, diastolic blood pressure, and a pulse rate per unit time of the measurement subject and a measurement date and time at predetermined time intervals.

The control unit 13 controls the communication unit 11 so as to transmit the measured blood-pressure-related information together with individual identification information of the measurement subject (or the blood pressure measuring instrument 10) to the server 30 via the user terminal 20 (or not via the user terminal 20). At this time, the control unit 13 may constantly transmit the blood-pressure-related information to the server 30 in real time or may collectively transmit pieces of blood-pressure-related information measured a plurality of times to the server 30. By reducing a frequency of communication performed by the communication unit 11, it is possible to reduce power consumption of the blood pressure measuring instrument 10.

The control unit 33 of the server 30 receives the blood-pressure-related information via the communication unit 31 and stores the blood-pressure-related information on the database of the storage unit 34.

The blood pressure measuring instrument 10, the user terminal 20, and the server 30 repeat the above-mentioned operation to accumulate the blood-pressure-related information of the measurement subject in the database of the storage unit 34.

Next, an example of operation in which the blood-pressure-related information accumulated in the database of the storage unit 34 is displayed on the blood-pressure-related information display apparatus will be described.

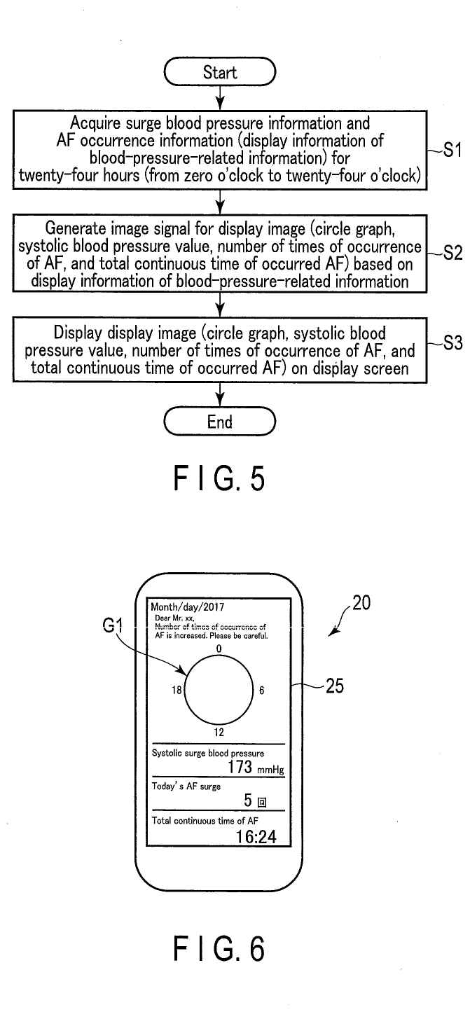

FIG. 5 is a flowchart showing a blood-pressure-related information display method according to an embodiment of the present invention. Note that, herein, an example where the user terminal 20 is used as the blood-pressure-related information display apparatus will be described.

For example, when the user operates the operation unit 22 so as to display blood-pressure-related information, the information acquisition unit 231 of the user terminal 20 transmits, via the communication unit 21, a signal for requesting display information of the blood-pressure-related information from the server 30. At this time, for example, a date at which the blood-pressure-related information to be displayed has been measured may be specified by the user, and an item to be displayed may be set in advance.

Further, the user can perform setting in advance so as to display the display information of the blood-pressure-related information at a predetermined time (e.g., at twenty-four o'clock every day or at ten o'clock on Saturday) of a predetermined date (or day of week), and, at the predetermined time of the predetermined date (or day of week), the information acquisition unit 231 may automatically (regardless of operation by the user) transmit a signal for requesting the display information of the blood-pressure-related information at the predetermined date from the server 30. For example, when the user performs setting in advance so as to display the blood-pressure-related information at twenty-four o'clock every day, the information acquisition unit 231 automatically transmits a signal for requesting display information of blood-pressure-related information measured from zero o'clock to twenty-four o'clock of that day from the server 30.

When the control unit 33 of the server 30 receives a signal for requesting display information of blood-pressure-related information from the user terminal 20 via the communication unit 31, for example, the control unit 33 reads blood-pressure-related information measured at a requested date from information stored on the database of the storage unit 34 and generates display information of the blood-pressure-related information to be displayed on the user terminal 20.

Note that the display information of the blood-pressure-related information includes, for example, a surge blood pressure value and a time at which the surge blood pressure has occurred (surge blood pressure information) which are based on the blood-pressure-related information of the measurement subject measured in a predetermined period of time (e.g., twenty-four hours from zero o'clock to twenty-four o'clock). Further, the display information of the blood-pressure-related information may include a time at which (or a period of time in which) atrial fibrillation of the measurement subject has occurred, the number of times of occurrence of atrial fibrillation, and a total continuous time of occurred atrial fibrillation (atrial fibrillation occurrence information) which are based on the blood-pressure-related information of the measurement subject measured in a predetermined period of time (e.g., twenty-four hours from zero o'clock to twenty-four o'clock). Note that the control unit 33 can determine, for example, whether atrial fibrillation occurs in the measurement subject based on information regarding a pulse rate and a pressure pulse wave of the measurement subject. Further, the control unit 33 can determine, for example, whether surge blood pressure occurs based on whether a blood pressure value of the measurement subject has been sharply increased in a unit time. When the control unit 33 generates display information of the requested blood-pressure-related information including the surge blood pressure information and the atrial fibrillation occurrence information, the control unit 33 transmits the display information to the user terminal 20 via the communication unit 31.

The information acquisition unit 231 of the user terminal 20 receives (acquires) the display information of the blood-pressure-related information from the server 30 via the communication unit 21 (step S1) and supplies the display information to the display processing unit 232.

The display processing unit 232 generates an image signal for displaying (a display image of) a circle graph G1, a systolic surge blood pressure value, the number of times of occurrence of atrial fibrillation surge, and a total continuous time of atrial fibrillation on the display screen of the display unit 25 based on the information received from the information acquisition unit 231 (step S2) and outputs the generated image signal to the display unit 25, thereby displaying the display image on the display screen (step S3).

FIG. 6 is a plan view illustrating an example where blood-pressure-related information is displayed on the display unit of the user terminal.

The display processing unit 232 can display, on the display screen of the display unit 25, for example, the circle graph G1 showing surge blood pressure values in time series along the circular time axis T, the systolic surge blood pressure value, the number of times of occurrence of atrial fibrillation surge, and the total continuous time of atrial fibrillation.

Further, when display information of past blood-pressure-related information is stored on the memory 24 of the user terminal 20, the display processing unit 232 may compare blood-pressure-related information of this time with the past blood-pressure-related information and display, on the display screen of the display unit 25, an evaluation result based on an item in which the health status of the measurement subject is improved, an item in which the health status is deteriorated, and the like.

FIG. 7 is an explanatory diagram illustrating a part of a circle graph included in a display of blood-pressure-related information.

The display processing unit 232 can display the surge blood pressure information as a circle graph G1, the circle graph G1 having a circular time axis T corresponding to twenty-four hours, having a time scale indicating elapse of a unit time in twenty-four hours clockwise along the time axis T, and including an aggregation of bars of a bar graph B that extends outward in a radial direction A of an arc of the time axis T and has height based on a surge blood pressure value per unit time.

The time axis T indicates, for example, a period of time from zero o'clock to twenty-four o'clock and has, for example, a circular shape in which numbers “0”, “6”, “12”, and “18” are displayed at positions corresponding to zero o'clock, six o'clock, twelve o'clock, and eighteen o'clock and zero o'clock and twenty-four o'clock are at the same position. Note that a time scale of the time axis T does not need to be displayed on the display screen, and, for example, a plurality of marks of the time scale may be reduced and only a part of the marks of the time scale may be displayed at predetermined intervals.

The bar graph B projects so as to have a mountain shape in the radial direction A from a position of the time scale of the time axis T corresponding to a time at which surge blood pressure has occurred, and height in the radial direction A is set corresponding to magnitude of a surge blood pressure value. Note that the height of the bar graph B does not need to be in proportion to the magnitude of the surge blood pressure value, and, for example, the height may be exaggeratedly displayed as to a high-risk surge blood pressure value. Further, the shape of the bar graph B is not limited to the above-mentioned shape and may be a shape projecting from the time axis T so as to be a substantially rectangular shape.

The display processing unit 232 may divide the bar graph B into a plurality of regions in a height direction as to the radial direction A and show the plurality of regions with different display colors and, for example, may gradually change display color of the bar graph B in accordance with a distance from the time axis T. The display processing unit 232 may, for example, change the display color of the bar graph B among a region within a range of the time axis T to a line L1, a region within a range of the line L1 to a line L2, a region within a range of the line L2 to a line L3, a region within a range of the line L3 to a line L4, and a region within a range outside the line L4. By gradually changing the display color of the bar graph B, magnitude of the surge blood pressure can be distinguished based on color. This allows the user to easily recognize the health status of the measurement subject.

Further, the display processing unit 232 may display a time at which (or a period of time in which) atrial fibrillation of the measurement subject has occurred on the circle graph G1. For example, the display processing unit 232 may display a plot (not illustrated) at a position of the time axis T corresponding to the time at which the atrial fibrillation has occurred and display the time at which (or the period of time in which) the atrial fibrillation has occurred together with a time at which surge blood pressure has occurred. Further, the display processing unit 232 may display, for example, a bar of the bar graph B corresponding to the time at which the atrial fibrillation of the measurement subject has occurred with color different from that of another bar of the bar graph B.

When, for example, the user performs operation on the operation unit 22 (e.g., operation of swiping on the touchscreen or operation of pressing the operation button) so as to change information to be displayed on the display screen, the display processing unit 232 can generate an image signal so as to change information to be displayed on the display screen and output the image signal to the display unit 25. With this, by operating the operation unit 22, the user can, for example, change information to be displayed together with the circle graph G1 and confirm a relation between the surge blood pressure information and the atrial fibrillation occurrence information.

(Effect)

As described above, a surge blood pressure value of the measurement subject and a time at which (or the period of time in which) atrial fibrillation has occurred are displayed by using the circle graph G1. Therefore, the measurement subject (or a third party such as a doctor or a family member) easily recognizes a tendency in which a surge blood pressure value and atrial fibrillation of the measurement subject occur (a period of time in which the surge blood pressure value occurs, a period of time in which the atrial fibrillation occurs, an interrelationship between surge blood pressure and atrial fibrillation, and the like). Therefore, it is possible to reduce a burden of health management of the measurement subject and assist in improving the health status of the measurement subject.

That is, according to the blood-pressure-related information display apparatus and the blood-pressure-related information display method of the present embodiment, it is possible to easily grasp the health status of the measurement subject based on a blood pressure value measured for twenty-four hours.

### Another Embodiment

Next, a blood-pressure-related information display apparatus and a blood-pressure-related information display method according to another embodiment will be described with reference to the drawings. Herein, an example of operation in which blood-pressure-related information accumulated in a database of a storage unit 34 is displayed on the blood-pressure-related information display apparatus will be described, and an example where a blood pressure measuring instrument 10 is used as the blood-pressure-related information display apparatus will be described. Note that, in the following description, configurations similar to those in the above-mentioned embodiment are denoted by the same reference signs, and description thereof will be omitted.

Operation in which blood-pressure-related information measured by the blood pressure measuring instrument 10 is accumulated in the storage unit 34 of a server 30 is similar to that in the above-mentioned embodiment, and therefore description thereof is herein omitted.

FIG. 8 is a flow-chart illustrating another example of the blood-pressure-related information display method according to an embodiment of the present invention.

For example, when the user operates the operation unit 12 so as to display blood-pressure-related information, the information acquisition unit 131 of the blood pressure measuring instrument 10 transmits, via the communication unit 11 (or the communication unit 11 or the user terminal 20), a signal for requesting display information of the blood-pressure-related information from the server 30. At this time, for example, a date at which the blood-pressure-related information to be displayed has been measured may be specified by the user, and an item to be displayed may be set in advance.

Further, the user can perform setting in advance so as to display the display information of the blood-pressure-related information at a predetermined time (e.g., at twenty-four o'clock every day or at ten o'clock on Saturday) of a predetermined date (or day of week), and, at the predetermined time of the predetermined date (or day of week), the information acquisition unit 131 may automatically (regardless of operation by the user) transmit a signal for requesting the display information of the blood-pressure-related information at the predetermined date from the server 30.

When the control unit 33 of the server 30 receives a signal for requesting display information of blood-pressure-related information from the blood pressure measuring instrument 10 (or the user terminal 20) via the communication unit 31, for example, similarly to the above embodiment, the control unit 33 reads blood-pressure-related information measured at a requested date from information stored on the database of the storage unit 34 and generates display information of the blood-pressure-related information to be displayed on the blood pressure measuring instrument 10.

When the control unit 33 generates the requested display information of the blood-pressure-related information, the control unit 33 transmits the display information to the blood pressure measuring instrument 10 via the communication unit 31 (or via the communication unit 31 and the user terminal 20).

The information acquisition unit 131 of the blood pressure measuring instrument 10 acquires the display information of the blood-pressure-related information via the communication unit 11 (step S4) and supplies the display information to the display processing unit 132.

Depending on the operation by the user and based on the information received from the information acquisition unit 131, the display processing unit 132 generates an image signal for displaying (a display image of) a circle graph G2, a systolic surge blood pressure value, the number of times of occurrence of atrial fibrillation surge, and a total continuous time of atrial fibrillation on the display screen of the display unit 16 (step S5) and outputs the generated image signal to the display unit 16, thereby displaying the display image on the display screen (step S6).

FIG. 9 is a plan view illustrating an example of a display of the blood-pressure-related information in the blood pressure measuring instrument.

The display processing unit 132 can display, on the display screen of the display unit 16, for example, the circle graph G2 showing surge blood pressure values in time series along the circular time axis T, the systolic surge blood pressure value, the number of times of occurrence of atrial fibrillation surge, and the total continuous time of atrial fibrillation. FIG. 9 shows an example where the circle graph G2, a systolic surge blood pressure value, and a date and time at which the systolic surge blood pressure value has occurred are displayed on the display screen.

The circle graph G2 has, similarly to the above circle graph G1, a circular time axis T corresponding to twenty-four hours, has a time scale indicating elapse of a unit time in twenty-four hours clockwise along the time axis T, and includes an aggregation of bars of a bar graph B that extends outward in a radial direction A of an arc of the time axis T and has height based on a surge blood pressure value per unit time.

The time axis T indicates, for example, a period of time from zero o'clock to twenty-four o'clock and has, for example, a circular shape in which numbers “0”, “6”, “12”, and “18” are displayed at positions corresponding to zero o'clock, six o'clock, twelve o'clock, and eighteen o'clock and zero o'clock and twenty-four o'clock are at the same position. Note that a time scale of the time axis T does not need to be displayed on the display screen, and, for example, a plurality of marks of the time scale may be reduced and only a part of the marks of the time scale may be displayed at predetermined intervals.

The bar graph B projects so as to have a mountain shape in the radial direction A from a position of the time scale of the time axis T corresponding to a time at which surge blood pressure has occurred, and height in the radial direction A (illustrated in FIG. 7) is set corresponding to magnitude of a surge blood pressure value. Note that the height of the bar graph B does not need to be in proportion to the magnitude of the surge blood pressure value, and, for example, the height may be exaggeratedly displayed as to a high-risk surge blood pressure value. Further, the shape of the bar graph B is not limited to the above-mentioned shape and may be a shape projecting from the time axis T so as to be a substantially rectangular shape.

The display processing unit 132 may divide the bar graph B into a plurality of regions in a height direction as to the radial direction A and show the plurality of regions with different display colors and, for example, may gradually change display color of the bar graph B in accordance with a distance from the time axis T. The display processing unit 132 may, for example, as illustrated in FIG. 7, change the display color of the bar graph B among a region within a range of the time axis T to a line L1, a region within a range of the line L1 to a line L2, a region within a range of the line L2 to a line L3, a region within a range of the line L3 to a line L4, and a region within a range outside the line L4.

Further, the display processing unit 132 may display a time at which (or a period of time in which) atrial fibrillation of the measurement subject has occurred on the circle graph G2. For example, the display processing unit 132 may display a plot (not illustrated) at a position of the time axis T corresponding to the time at which the atrial fibrillation has occurred and display the time at which (or the period of time in which) the atrial fibrillation has occurred together with a time at which surge blood pressure has occurred. Further, the display processing unit 132 may display, for example, a bar of the bar graph B corresponding to the time at which the atrial fibrillation of the measurement subject has occurred with color different from that of another bar of the bar graph B.

Further, the display processing unit 132 may, for example, display information regarding surge blood pressure displayed on the circle graph G2 and another information displayed on the display screen of the display unit 16 in association with each other by flickering a bar of the bar graph B corresponding to the systolic surge blood pressure displayed on the display screen of the display unit 16 or displaying the bar of the bar graph B with color different from that of another bar of the bar graph B.

When, for example, the user performs operation on the operation unit 12 (e.g., operation of swiping on the touchscreen or operation of pressing the operation button) so as to change information to be displayed on the display screen, the display processing unit 132 can generate an image signal so as to change information to be displayed on the display screen and output the image signal to the display unit 16. With this, by operating the operation unit 12, the user can, for example, change information to be displayed together with the circle graph G2 and confirm a relation between the surge blood pressure information and the atrial fibrillation occurrence information.

Note that, in a case where the blood pressure measuring instrument 10 further includes a speaker (not illustrated), the measurement subject may be notified of display by an alarm when blood-pressure-related information is displayed on the blood pressure measuring instrument 10. This makes it possible to prevent the measurement subject from missing the information displayed on the blood pressure measuring instrument 10, and therefore it can be expected that the measurement subject habitually checks his/her health status.

(Effect)

As described above, a surge blood pressure value of the measurement subject and a time at which (or the period of time in which) atrial fibrillation has occurred are displayed by using the circle graph G2. Therefore, the measurement subject himself/herself easily recognizes a tendency in which a surge blood pressure value and atrial fibrillation of the measurement subject occur (a period of time in which the surge blood pressure value occurs, a period of time in which the atrial fibrillation occurs, an interrelationship between surge blood pressure and atrial fibrillation, and the like). Therefore, it is possible to reduce a burden of health management of the measurement subject himself/herself and assist in improving the health status of the measurement subject himself/herself.

Further, as described above, blood-pressure-related information is displayed on the display unit 16 of the blood pressure measuring instrument 10, and therefore the measurement subject can check his/her health status by using the blood pressure measuring instrument 10 that is always worn on a measurement part in order to measure blood pressure. This makes it possible to further improve convenience for the measurement subject.

That is, according to the blood-pressure-related information display apparatus and the blood-pressure-related information display method of the present embodiment, it is possible to provide the blood-pressure-related information display apparatus and the blood-pressure-related information display method that facilitate a grasp of the health status of the measurement subject based on blood-pressure-related information that is continuously measured for a long period of time.

Note that, in each of the above-mentioned embodiments, there has been described the blood-pressure-related information display apparatus and the blood-pressure-related information display method that display surge blood pressure information and atrial fibrillation occurrence information of the measurement subject measured for twenty-four hours. However, the information to be displayed on the display unit is not limited to information for twenty-four hours. For example, the blood-pressure-related information display apparatus may be configured to display surge blood pressure information and atrial fibrillation occurrence information of the measurement subject only in a period of time set by the user. In that case, the bar graph B or the like may be displayed only in a period of time corresponding to the time scale of a part of the time axis T on the assumption that the time axis T indicates twenty-four hours, and a start time, an end time, and the time scale of the time axis T may be adjusted in accordance with a period of time in which predetermined information is displayed.

Further, in the above-mentioned embodiments, the blood-pressure-related information display apparatus requests display information of blood-pressure-related information from the server, and therefore the blood-pressure-related information display apparatus obtains the display information. However, for example, when it is determined that the health status of the measurement subject is deteriorated, the display information of the blood-pressure-related information may be transmitted to the blood-pressure-related information display apparatus even if the display information is not requested by the blood-pressure-related information display apparatus. With this, the user can early recognize deterioration in the health status of the measurement subject, and therefore it is possible to perform health management of the measurement subject more effectively. Note that operation of the blood-pressure-related information display apparatus at this time is similar to that in the above-mentioned embodiments, except that the display information of the blood-pressure-related information is not requested.

Note that in the above embodiment, the control unit 13 can be configured to determine, for example, whether atrial fibrillation occurs in the measurement subject based on information regarding a pulse rate and a pressure pulse wave of the measurement subject. Further, the control unit 13 can be configured to determine, for example, whether surge blood pressure occurs based on whether a blood pressure value of the measurement subject has been sharply increased in a unit time.

Note that in the above embodiment, the control unit 23 can be configured to determine, for example, whether atrial fibrillation occurs in the measurement subject based on information regarding a pulse rate and a pressure pulse wave of the measurement subject. Further, the control unit 23 can be configured to determine, for example, whether surge blood pressure occurs based on whether a blood pressure value of the measurement subject has been sharply increased in a unit time.

In those cases, the blood-pressure-related information display apparatus requesting the display information of the blood-pressure-related information from the server can be omitted.

The present invention is not limited to the above-mentioned embodiments and may be embodied with the components modified without departing from the scope of the invention. Further, various inventions can be made by appropriately combining a plurality of constituent elements disclosed in the above-mentioned embodiments. For example, it is possible to remove some of the components shown in the embodiments. Further, components indifferent embodiments may be appropriately combined.

Some or all of the above embodiments may also be described as attached below, but is not limited to the following.

### (Appendix 1)

A blood-pressure-related information display apparatus that displays information regarding blood pressure of a measurement subject on a display screen, the blood-pressure-related information display apparatus including:

a hardware processor and a memory,

wherein the hardware processor is configured to acquire surge blood pressure information of the measurement subject; and

to display the surge blood pressure information as a circle graph on the display screen, the circle graph having a circular time axis corresponding to twenty-four hours, having a time scale indicating elapse of a unit time in the twenty-four hours clockwise along the time axis, and including an aggregation of bars of a bar graph that extends outward in a radial direction of an arc of the time axis and has height corresponding to a value based on the surge blood pressure information per the unit time.

### (Appendix 2)

A blood-pressure-related information display method of displaying information regarding blood pressure of a measurement subject on a display screen, the blood-pressure-related information display method including:

acquiring surge blood pressure information of the measurement subject using at least one or more hard processors; and

displaying, using at least one or more hard processors, the surge blood pressure information as a circle graph on the display screen, the circle graph having a circular time axis corresponding to twenty-four hours, having a time scale indicating elapse of a unit time in the twenty-four hours clockwise along the time axis, and including an aggregation of bars of a bar graph that extends outward in a radial direction of an arc of the time axis and has height corresponding to a value based on the surge blood pressure information per the unit time.

1. A blood-pressure-related information display apparatus that displays information regarding blood pressure of a measurement subject on a display screen, the blood-pressure-related information display apparatus comprising:
an information acquisition unit that acquires surge blood pressure information of the measurement subject; and
a display processing unit that displays the surge blood pressure information as a circle graph on the display screen, the circle graph having a circular time axis corresponding to twenty-four hours, having a time scale indicating elapse of a unit time in the twenty-four hours clockwise along the time axis, and including an aggregation of bars of a bar graph that extends outward in a radial direction of an arc of the time axis and has height corresponding to a value based on the surge blood pressure information per the unit time,
wherein the surge blood pressure information includes a systolic surge blood pressure value of the measurement subject in the twenty-four hours, and
the display processing unit displays a bar of the bar graph corresponding to the systolic surge blood pressure value with color different from color of another bar of the bar graph.

2. The blood-pressure-related information display apparatus according to claim 1, wherein
the display processing unit divides the bar graph into a plurality of regions in the radial direction and shows the plurality of regions with different display colors.

3. The blood-pressure-related information display apparatus according to claim 1, wherein
the information acquisition unit further acquires atrial fibrillation occurrence information of the measurement subject, and
the display processing unit displays a plot on the time scale corresponding to a time at which atrial fibrillation of the measurement subject has occurred.

4. The blood-pressure-related information display apparatus according to claim 1, further comprising:
a strip that is to be worn on a measurement part of the measurement subject;
a sensor unit that is provided so that at least a part of the sensor unit is brought into contact with the measurement part when the strip is worn on the measurement part;
a blood-pressure-information measurement unit that adjusts pressure to be applied from the sensor unit to the measurement part and measures systolic blood pressure and diastolic blood pressure of the measurement subject based on the pressure; and
a communication unit that transmits values measured by the blood-pressure-information measurement unit to outside.

5. A blood-pressure-related information display method of displaying information regarding blood pressure of a measurement subject on a display screen, the blood-pressure-related information display method comprising:
acquiring surge blood pressure information of the measurement subject, the surge blood pressure information including a systolic surge blood pressure value of the measurement subject in the twenty-four hours; and
displaying the surge blood pressure information as a circle graph on the display screen, the circle graph having a circular time axis corresponding to twenty-four hours, having a time scale indicating elapse of a unit time in the twenty-four hours clockwise along the time axis, including an aggregation of bars of a bar graph that extends outward in a radial direction of an arc of the time axis dud has height corresponding to a value based on the surge blood pressure information per the unit time, and displaying a bar of the bar graph corresponding to the systolic surge blood pressure value with color different from color of another bar of the bar graph.
